(19) United States
(12) Patent Application Publication
(10) Pub. No.: US 2003/0031690 A1
(43) Pub. Date: Feb. 13, 2003

(54) Cosmetic composition for preventing and treating acnes containing cryptotanshinone

(76) Inventors: Nae-Gyu Kang, Daejeon (KR); Jung-Ah Kim, Daejeon (KR); Young-Sook Song, Daejeon (KR); Jhong-Tae Lee, Daejeon (KR); Wan-Goo Cho, Daejeon (KR); Seh-Hoon Kang, Daejeon (KR)

Correspondence Address: MCDERMOTT, WILL & EMERY, 600 13th Street, N.W., Washington, DC, 20005-3096, US

(21) Appl. No.: 09/941,928

(22) Filed: Aug. 30, 2001

(30) Foreign Application Priority Data
Mar. 26, 2001 (KR) 2001-15756

(51) Int. Cl. A61K 7/06; A61K 9/14

(52) U.S. Cl. 424/401; 424/70.1

(57) ABSTRACT

A cosmetic composition for preventing and treating acne, contains cryptotanshinone as an effective component. The cosmetic composition containing cryptotanshinone can be safely used without any side effect on skin, and further, can very effectively control the amount of sebum secretion due to inhibition of 5 α-reductase activation, and has superior anti-bacterial and anti-inflammation ability.

### BACKGROUND OF THE INVENTION

1. Field of the Invention

The present invention relates to cosmetic compositions for preventing and treating acne, and more particularly, to cosmetic compositions for preventing and treating acne containing cryptotanshinone as an effective component.

2. Description of the Related Art

Acne is chronic inflammatory disease of the hair sebaceous gland, breaking out on the face, breast, or neck of young people generally at about the age of puberty. The same terminology is used for a lesion forming comedones, papule, pustule, cystoma, or nodule having the potential of leaving a scar in a serious case. When the skin damage due to acne is serious, people can be mentally injured as a result, for example, suffering from hypochondria or avoiding personal relations.

The cause of outbreak of acne has not been clearly determined yet. However, if genetic and the environmental factors are excluded, the following three factors are acknowledged as causes of acne.

A first cause is abnormal over-secretion of sebum. The over-secretion of sebum is closely related to the male hormone, testosterone. Testosterone is known to promote the secretion of sebum by being converted into dihydrotestosterone (DHT) by 5 α-reductase in skin cell, thereby increasing the activity of the sebum secretion cell (Rosenfield R L and Deplewski D. Role of androgen in the developmental biology of the pilosebaceous unit. The American Journal of Medicine 1995; 16:80s-88s).

A second cause is the closing of a pore by over-keratinization of the wall of a hair pouch. The sebum generated should smoothly be eliminated outside the skin through the pores. However, when the pore is closed due to over-keratinization of the wall of hair pouch, smooth elimination of sebum is restrained. Accordingly, the sebum becomes stagnated in the hair pouch, which generates fine comedones. This is the first stage of acne.

A third cause is that an acne lesion may become more serious because the sebum becomes stagnated in the hair pouch and promotes the growth of anaerobic bacteria residing inside the hair pouch, i.e. Propionibacterium acnes. As the acne bacteria inside the sebaceous gland proliferate, triglyceride composing the sebum is converted into free fatty acid that functions as an irritant or as a comedogenic material. Accordingly, acne is promoted to generate, and be accompanied by, inflammation (Sergio Nacht, Cosmetics Toiletries, 101, 47-55, 1986; Wei-Li Lee, et a., Infection and Immunity, 35, 71-78, 1982).

Accordingly, on the basis of such causes of acne outbreak, the effects of treating and alleviating acne are measured by the effects of controlling the secretion of sebum, restraining the over-keratinization of the of hair pouch wall, anti-bacterially functioning against acne bacteria, and alleviating inflammation generated by the acne bacteria (Madli Puhvel, Cosmetic Toiletries, 98, 117-119, 1983; Kathy Perisho, et al., J. of Invest. Dermatol., 90, 350-353, 1988; Hyeyoung Kim, et al., Korean J. Ginseng Sci., 14, 391-398, 1990; R. A. Bojar, et al., British J. of Dermatology, 132, 204-208, 1995; A. M. Kligman, et al., British J. of Dermatology, 100, 699-702, 1979).

It is known to treat acne by a method of making the secretion of sebum to be smooth by restraining over-keratinization of the hair pouch wall or eliminating the part excessively keratinized of the hair pouch wall by using salicylic acid or retinoic acid of vitamin A derivative, and by a method of eliminating the skin keratin and alleviating the inflammation by using benzoyl peroxide, triclosan, sulfur, or tea tree oil having antibacterial effect.

However, when salicylic acid is used, the remedial effect on acne is insignificant, and furthermore, the same can cause skin flare or edema. In addition, retinoic acid has an effect of restraining the hair pouch wall from being over-keratinized, but the same can cause contact dermatitis, erythema, dryness and exfoliation of skin. Using benzoyl peroxide or triclosan requires attention since it is possible to cause side effects such as serious erythema as well as allergic contact dermatitis and scarrin. On the other hand, the side effect decreases on using azelaic acid relatively recently introduced, but the remedial effect also is decreased in comparison with that of conventional acne treatment agents.

### SUMMARY OF THE INVENTION

An object of the present invention is to solve the above problems and to provide cosmetic compositions for preventing and treating acne that can be safely used without side effect, controlling the amount of sebum secretion through inhibiting the activity of 5 α-reductase, and having superior antibacterial and anti-inflammatory effects.

To achieve the above objects, the present invention provides cosmetic compositions having a characteristic of containing cryptotanshinone as an effective component. Cryptotanshinone causes little stimulus adverse to skin, and furthermore, can effectively prevent and treat acne by inhibiting the activity of 5α-reductase, thereby decreasing the amount of sebum secretion and impeding the growth of Propionibacterium acnes to alleviate inflammation. Accordingly, if cryptotanshinone is added to compositions for various types of cosmetics such as toner, lotion, cream, foundation, essence, gel, pack, foam cleansing, or soap, the effect of preventing and treating acne as well as basic function as cosmetics can be obtained.

On forming the cosmetic composition of the present invention, content of cryptotanshinone is preferably 0.001-10weight % on the basis of total weight.

In addition, the cosmetic composition of the present invention can contain further material for restraining the hair pouch wall from being over-keratinized such as salicylic acid, retinoic acid, retinoic acid derivative, or benzoyl peroxide.

### DETAILED DESCRIPTION AND PREFERRED EMBODIMENTS OF THE INVENTION

The detailed description of the cosmetic composition according to the present invention is provided hereinafter.

Cryptotanshinone is material belonging to tanshinone class and its structural formula is as follows.

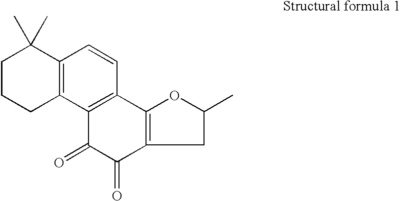

Cryptotanshinone can be obtained by a process of chemical synthesis or by a process of extraction from plants. In the case of chemical synthesis, only a small amount of cryptotanshinone that could be obtained in a laboratory can be obtained and the synthesizing efficiency is deteriorated. Accordingly, it is preferable to obtained cryptotanshinone from plants by extraction. For example, cryptotanshinone can be extracted from roots of the perennial herbal plant Salvia militiorrhira Bge.

According to the results of various clinical tests mentioned below, cryptotanshinone stimulates skin very slightly in contrast with conventional medicines for treating acne. Further, cryptotanshinone works very effectively to prevent and treat acne by inhibiting 5 α-reductase, thereby decreasing the amount of sebum secretion, and impeding the growth of Propionibacterium acnes, thereby alleviating inflammation.

Accordingly, if cryptotanshinone is added to compositions of various types of cosmetics such as toner, lotion, cream, foundation, essence, gel, pack, foam cleansing, or soap, the effect of preventing and treating acne, as well as basic function as cosmetics can be obtained. Furthermore, if material for restraining the hair pouch wall from being over-keratinized, such as salicylic acid, retinoic acid, retinoic acid derivative or benzoyl peroxide are added to cryptotanshinone-containing compositions, more effective prevention and treatment of acne can be expected.

On forming the cosmetic composition of the present invention, content of cryptotanshinone is preferably 0.001-10 weight % on the basis of total weight. When the content of cryptotanshinone is less than 0.001 weight %, the effect of treating acne maybe decreased in comparison to the expected effect, and when over 10 weight %, there can be difficulty in compounding the cosmetic composition. Therefor, the content of cryptotanshinone is more preferably in the range of about 0.001-2 weight %.

The concrete description of the present invention referring to the experimental examples and embodiments is provided hereinafter. However, the embodiments according to the present invention can be modified in various ways and the claims of the present invention should not be understood to be restricted to the embodiments described below. The embodiments of the present invention are provided to more concretely describe the present invention to a person who has ordinary knowledge in this technical field.

### ASSESSMENT OF EFFECT OF RESTRAINING SEBUM SECRETION OF CRYPTOTANSHINONE EXPERIMENTAL EXAMPLE 1

The effect of restraining sebum secretion is verified by the following experiment showing the inhibiting effect of cryptotanshinone on 5 α-reductase.

30 l of Phosphate Buffered Saline (PBS/pH 6.6) is mixed with 101 of dithiothreitol, NADPH, and testosterone, into which radioactive isotopes are combined (3H-1, 2, 4, 5, 6, 7-testosterone), 401 of extract liquid of enzyme 5 α-reductase, and 20 l of cryptotanshinone having 0.001% of concentration. Next, the composition mentioned above is left at the normal temperature for 10 minutes so that the materials in the composition react to each other, and then the amount of converted dihydrotestosterone are measured by HPLC and a measuring instrument of radioisotope. According to the following formula, the inhibition rate (%) of cryptotanshinone against 5 α-reductase is obtained and Table 1 shows the result.

Inhibition rate (%)=(conversion rate from testosterone to dihydrotestosterone with no test material)×100/(conversion rate from testosterone to dihydrotestosterone with test material).

### Experimental Examples 2-4

The experiments are conducted in the same manner as experimental example 1, except for changing the concentration of cryptotanshinone as described in Table 1.

TABLE 1

| | Concentration of | |
|---|---|---|
| | cryptotanshinone (%) | Inhibition rate (%) |
| Experimental example 1 | 0.001 | 19.1 |
| Experimental example 2 | 0.01 | 36.0 |
| Experimental example 3 | 0.1 | 45.9 |
| Experimental example 4 | 1.0 | 100 |

As shown in Table 1, cryptotanshinone is very effective to inhibit the activity of 5 α-reductase.

### Assessment of Antibacterial Ability of Cryptotanshinone Against Propionibacterium acnes

### Experimental Example 5

To measure the antibacterial ability of cryptotanshinone against Propionibacterium acnes (P. acnes hereinafter), P. acnes kept in a cold storage in original state was cultivated on a BHI (Brain Heart Infusion) solid flat medium three days before the beginning of experiment. On the day of experiment, the P. acnes previously cultivated was inoculated into BHI solid flat medium containing the predetermined concentration of cryptotanshinone. Three days later after that, the minimum inhibition concentration (MIC) is obtained by measuring the number of P. acnes. The result is shown in Table 2.

### Comparative Example 1

The experiment was conducted in the same manner as experimental example 5, except for using benzoyl peroxide instead of cryptotanshinone.

TABLE 2

| | Testing material | MIC (mole) |
|---|---|---|
| Experimental example 5 | Cryptotanshinone | 100-500 ppm |
| Comparative example 1 | Benzoyl peroxide | 500-1000 ppm |

Referring to Table 2, cryptotanshinone can more effectively inhibit the growth of P. acnes than benzoyl peroxide, which is used as an effective composition for a conventional acne treatment medicine.

### Assessment of Anti-Inflammation Effect of Cryptotanshinone Experimental Example 6

When inflammation is generated due to P. acnes, become more serious. Accordingly, the effect of alleviating the inflammation due to acne is verified by assessing the anti-inflammatory effect of cryptotanshinone. The experiment is conducted by a method using arachidonic acid (A. Crummey, G. P. Harper, E. A. Boyle and F. R. Mangan. Inhibition of arachidonic acid-induced ear oedema as a model for assessing topical anti-inflammatory compound. Agents and Actions 1987; 20:69-76).

After washing both ears of mice with ethanol before applying the agent, 201 o f cryptotanshinone having a concentration of 0.3% is applied to the ears of mice belonging to an agent application group once a day for four days, while 201 o f ethanol is applied to mice in a comparative group. One hour after the last application, ethanol is applied to the left ears of mice and 2 mg/ear of arachidonic acid is applied to the right ears of mice. One hour after that, the degree of ear edema is repeatedly measured by micrometer three times for each ear. The left ears of mice in the agent application group are used as a comparative group for measuring the degree of inflammation being induced by the agent.

The anti-inflammatory effect is assessed by the degree that the edema in the agent application group is restrained by cryptotanshinone on the basis of the comparative group to which arachidonic acid is applied. The result is shown in Table 3. The number of mice used for each group is seven and the value is an average of those of 7 mice.

### Comparative Examples 2-3

The experiments are conducted in the same manner as experimental example 6, except for respectively using stearyl glycyrrhetinate and indomethacin instead of cryptotanshinone.

TABLE 3

| | | | | Thickness of ear | | |
|---|---|---|---|---|---|---|
| Agent | | | | Before | After | |
| application | | Concentration | | applying | applying | Restraint rate |
| group | Agent | (%) | Solvent | agent | agent | (%) |
| Comparative | Arachidonic | 2 mg/ear | Ethanol | 272 | 493 | — |
| group | acid | | | | | |
| Experimental | Cryptotanshin | 0.3 | Ethanol | 271 | 437 | 24.9 |
| example 6 | one | | | | | |
| Comparative | Stearyl | 0.3 | Ethanol | 268 | 454 | 15.8 |
| example 2 | Glycyrrhetinate | | | | | |
| Comparative | Indomethacin | 0.3 | ethanol | 273 | 391 | 46.6 |
| example 3 | | | | | | |

*Restraint rate (%) = (A − B)/A × 100
A: Change of average thickness of ear in the comparative group (Thickness of ear to which arachidonic acid is applied - thickness of ear to which none is applied)
B: Change of average thickness of ear in the agent application group (Thickness of ear to which the agent is applied - thickness of ear to which none is applied)

As shown in Table 3, cryptotanshinone has superior inflammation restraint rate of 24.9%. This value is higher than that of stearyl glycyrrhetinate of comparative example 2, which is known to have good anti-inflammatory activity and corresponds to about ½ of that of Indomethacin, a medicament having superior anti-inflammation effect. Further, the test of significance to increase rate of edema showed 99% of significant level.

From the above experimental examples, it is verified that cryptotanshinone has a superior effect on inhibition of 5 α-reductase activation and anti-bacterial and anti-inflammation functions against P. acnes.

Hereinafter, the effects of restraining sebum secretion, alleviating comedones, and improving the state of acne in vivo of cosmetic compositions containing cryptotanshinone in the form of gel, lotion, essence, or cream are assessed. The experiments using toner, foundation, or pack have been conducted and a similar effect is obtained, have been omitted here.

Embodiment 1 and Comparative case 1

A gel is manufactured with components and contents as described in Table 4.

TABLE 4

| | Embodiment | Comparative case |
|---|---|---|
| Name of material | 1 (weight %) | 1 (weight %) |
| Glycerin | 3.0 | 3.0 |
| Dipropylene glycol | 5.0 | 5.0 |
| Carboxy vinyl polymer | 0.2 | 0.2 |
| Ethanol | 10.0 | 10.0 |
| Polyoxyethylene | 0.3 | 0.3 |
| hardened caster-oil | | |
| Triethanolamine | 0.2 | 0.2 |
| Cryptotanshinone | 0.1 | — |
| Preservative | q.s | q.s |
| Aromatic material | q.s | q.s |
| Pigment | q.s | q.s |
| Refined water | to 100 | to 100 |

Embodiment 2 and Comparative case 2

A lotion is manufactured with components and contents as described in Table 5.

TABLE 5

| Name of | Embodiment | Comparative case |
|---|---|---|
| material | 2 (weight %) | 2 (weight %) |
| Glyceryl | 2.0 | 2.0 |
| stearate | | |
| Cetearyl | 0.8 | 0.8 |
| alcohol | | |
| Stearic acid | 1.0 | 1.0 |
| Squalane | 8.0 | 8.0 |
| Refined | 4.0 | 4.0 |
| jojoba oil | | |
| 2-Octyl- | 2.0 | 2.0 |
| dodecanol | | |
| Polysorbate 60 | 1.0 | 1.0 |
| Sorbitanses- | 0.5 | 0.5 |
| quiolate | | |
| Glycerin | 5.0 | 5.0 |
| Carbomer | 0.2 | 0.2 |
| Triethanol- | 0.3 | 0.3 |
| amine | | |
| Crypto- | 0.5 | — |
| tanshinone | | |
| Preservative | q.s | q.s |
| Fragrance | q.s | q.s |
| Pigments | q.s | q.s |
| Water | to 100 | to 100 |

Embodiment 3 and Comparative Case 3

An essence is manufactured with components and contents as described in Table 6.

TABLE 6

| | Embodiment | Comparative case |
|---|---|---|
| Name of material | 3 (weight %) | 3 (weight %) |
| Denatured ethanol | 6.0 | 6.0 |
| Glycerin | 5.0 | 5.0 |
| Poly acrylic acid | 0.2 | 0.2 |
| 1,3-butylene glycol | 10.0 | 10.0 |
| Hydroxy ethyl cellulose | 0.1 | 0.1 |
| Carboxylic | 0.1 | 0.1 |
| ethylene cellulose | | |
| Triethanolamine | 0.2 | 0.2 |
| Ethylene oxide | 0.3 | 0.3 |
| Cryptotanshinone | 0.3 | — |
| Preservative | q.s | q.s |
| Fragrance | q.s | q.s |
| Pigments | q.s | q.s |
| Water | to 100 | to 100 |

Embodiment 4 and Comparative Case 4

A cream is manufactured with components and contents as described in Table 7.

TABLE 7

| | Embodiment | Comparative case |
|---|---|---|
| Name of material | 4 (weight %) | 4 (weight %) |
| Microcrystalline wax | 0.5 | 0.5 |
| Cetearyl alcohol | 3.0 | 3.0 |
| Stearic acid | 2.0 | 2.0 |
| Squalane | 5.0 | 5.0 |
| Stearic acid glycerin | 2.0 | 2.0 |
| 2-Octyldodecanol | 3.0 | 3.0 |
| Polysorbate 60 | 1.5 | 1.5 |
| Sorbitansesquiolate | 0.5 | 0.5 |
| Liquid paraffin | 8.0 | 8.0 |
| 2-Octyldodecyl myristate | 4.0 | 4.0 |
| Glycerin | 6.0 | 6.0 |
| Dipropylene glycol | 5.0 | 5.0 |
| Cryptotanshinone | 0.5 | — |
| Preservative | q.s | q.s |
| Aromatic material | q.s | q.s |
| Pigments | q.s | q.s |
| Water | to 100 | to 100 |

First Stimulus Test to Human Skin (Closed Patch Test)

To verify that the cosmetics containing cryptotanshinone according to the above embodiments are harmful, a first stimulus test on human skin was made through a Human patch test as follows.

The test was carried out with fifty healthy adult men and women according to CTFA (The Cosmetic, Toiletry and Fragrance Association Inc. Washington, D.C., 20036,1991) guideline.

20 l of each cosmetic material according to the embodiments and the comparative cases was dropped in a Finn Chamber, and then, the same was put on a tested part of human body and fixed by an adhesive tape. Twenty-four hours later, the patch was removed. Four (4) hours later, skin reaction of the tested part was observed with the naked eye according to the following criterion and the results are shown in Table 8.

TABLE 8

| | Number | | | | | |
|---|---|---|---|---|---|---|
| Testing | of | Result of Interpretation | | | | Degree of |
| material | subject | + + | + | + − | − | stimulus |
| Embodiment 1 | 50 | 0 | 0 | 1 | 49 | 0.02 |
| Comparative | 50 | 0 | 0 | 3 | 47 | 0.06 |
| case 1 | | | | | | |
| Embodiment 2 | 50 | 0 | 0 | 1 | 49 | 0.02 |
| Comparative | 50 | 0 | 0 | 2 | 48 | 0.04 |
| case 2 | | | | | | |
| Embodiment 3 | 50 | 0 | 0 | 0 | 50 | 0.00 |
| Comparative | 50 | 0 | 0 | 1 | 49 | 0.02 |
| case 3 | | | | | | |
| Embodiment 4 | 50 | 0 | 0 | 0 | 50 | 0.00 |
| Comparative | 50 | 0 | 0 | 2 | 48 | 0.04 |
| case 4 | | | | | | |

Interpreting Criterion
: There is no special symptom such as erythema
+ −: To redden a bit more than surrounding
+: To redden remarkably more than surrounding
+ +: To redden seriously more than surrounding and to swell up
Degree of stimulus = [{number of (+−) × 1} + {number of (+) × 2} + {number of (++) × 3}]/number of subject

As shown in Table 8, the cosmetic materials of the embodiments 1-4, containing cryptotanshinone show a lower degree of stimulus than that of the cosmetic materials of the comparative cases 1-4, not containing cryptotanshinone. While not wishing to be bound by theory, it is presumed that this result is due to the superior inflammation alleviation effect of cryptotanshinone, as verified in the above-described experiment regarding its anti-inflammation effect. Accordingly, there it has been verified that cryptotanshinone is a very safe material for skin when the same is used for the cosmetics.

Test for Assessing Restraint Effect of Sebum Secretion

To assess the restraint effect of sebum secretion, forty health grown-up men and women are chosen at random and a sebum tape (Sebutape, CuDerm Corp., TX, USA) is used. A method for testing is as followed.

First, the speed of normal sebum secretion on a forehead portion of a subject was measured by a sebum tape, and then, the subjects applied the cosmetic materials of the embodiments 1-3 and the comparative cases 1-3 for three weeks. After three weeks, the speed of sebum secretion on the same portion was measured again by the sebum tape. The sebum tape was adhered on a forehead portion for 30 minutes and removed. Next, the amount of sebum left on the sebum tape was measured by an image analyzer and the speed of sebum secretion was calculated. The results are shown in Table 9. (Unit of speed of sebum secretion is μg/Cm²/min and it means the amount of sebum secreted per unit time and unit area).

TABLE 9

| | Speed of | (Comparative case- |
|---|---|---|
| | sebum secretion | embodiment)/comparative |
| Testing material | (μg/Cm²/min) | case × 100 |
| Embodiment 1 | 0.89 | 38.6% |
| Comparative case 1 | 1.45 | |
| Embodiment 2 | 0.82 | 40.6% |
| Comparative case 2 | 1.38 | |
| Embodiment 3 | 0.92 | 39.5% |
| Comparative case 3 | 1.52 | |

As shown in Table 9, in the case where the cosmetic materials of the embodiments 1-3 were applied shows lower speed of sebum secretion than that of a case that the cosmetic materials of the comparative cases 1-3. This means that the amount of secreted sebum is reduced too. While not wishing to be bound by theory, this result is believed to be caused by the inhibition of 5 α-reductase activity by cryptotanshinone as shown in the above-described experiment.

Assessment of Effect of Restraining Comedone Growth Using Ears of Rabbit

The effect of restraining comedones growth is assessed through a method using rabbit ear, widely known as a method for assessing the effect on acne (A. Am. Kligman, et al., British Journal of Dermatology, 100, 699-702, 1979).

First, 0.5 l of each cosmetic material of the embodiments 1-3 were applied to one ear of rabbit and the same amount of each cosmetic material of the comparative cases 1-3 on the other ear thereof. Five (5) hours later, 0.5 l o f isopropyl myristate was applied to each ear. This treatment was repeated every day for two weeks. After that, the effect of restraining comedones growth was assessed with the naked eye and a result classified into 7 levels of ‘0’, ‘0.5’, ‘1’, ‘1.5’, ‘2’, ‘2.5’, and ‘3’ according to the following criterion is shown in table 10. Further, a whole mount was made on the ear of rabbit and the area (mm²) of comedones was measured and assessed by an image analyzer. The results are shown in Table 11. The number of rabbits used for each experiment were respectively 7.

TABLE 10
Criterion for assessing with the naked eye

| | |
|---|---|
| 0: | There is no difference from that of the comparative group. |
| 0.5: | Symptom somewhere between ‘0’ and ‘1’ is observed. |
| 1: | Congestion, capillary hemorrhage, and slight pore keratosis and |
| | hypertrophy are observed. |
| 1.5: | Symptom somewhere between ‘1’ and ‘2’ is observed. |
| 2: | Medium degree of pore keratosis and hypertrophy are observed. |
| 2.5: | Symptom somewhere between ‘2’ and ‘3’ is observed. |
| 3: | Serious pore keratosis and hypertrophy, and typical comedones are |
| | observed. |

| | Assessment with | (Comparative case- |
|---|---|---|
| | the naked | embodiment)/comparative |
| Testing material | eye | case × 100 |
| Embodiment 1 | 1.52 | 39.4% |
| Comparative case 1 | 2.51 | |
| Embodiment 2 | 1.64 | 42.5% |
| Comparative case 2 | 2.85 | |
| Embodiment 3 | 1.66 | 40.5% |
| Comparative case 3 | 2.79 | |

TABLE 11

| | Comparison | (Comparative case- |
|---|---|---|
| | of comedones | embodiment)/comparative |
| Testing material | area | case × 100 |
| Embodiment 1 | 1.340 | 36.4% |
| Comparative case 1 | 2.107 | |
| Embodiment 2 | 1.398 | 39.7% |
| Comparative case 2 | 2.318 | |
| Embodiment 3 | 1.438 | 37.5% |
| Comparative case 3 | 2.301 | |

As shown in Tables 10 and 11, it has been verified that the cosmetic materials of the embodiments 1-3 have superior effect of restraining comedones growth compared to that of the cosmetic materials of the comparative cases 1-3. Accordingly, there one can reasonably conclude that the cosmetic material containing cryptotanshinone is very effective in preventing acne.

Assessment of Effect of Alleviating Comedones Using Ears of Rabbit

The effect of alleviating comedones of cosmetic material containing cryptotanshinone was assessed by the foregoing method for assessing the effect of restraining comedones growth using ears of rabbit.

First, 0.5 l of isopropyl myristate was applied to each ear for two weeks to induce comedones, and then, 0.5 l of each cosmetic material of the embodiments 1-3 were applied to one ear and the same amount of each cosmetic material of the comparative cases 1-3 was applied to the other ear for two weeks. After that, the effect of alleviating comedones was assessed with the naked eye and a results classified into 7 levels of ‘0’, ‘0.5’, ‘1’, ‘1.5’, ‘2’, ‘2.5’, and ‘3’ according to the following criterion are shown in Table 12. Further, a whole mount is made on the ear of rabbit and the area (mm²) of comedones is measured and assessed by an image analyzer. The results are shown in Table 13. The numbers of rabbit used for experiments are respectively 7.

TABLE 12
Criterion for assessing with the naked eye

| | |
|---|---|
| 0: | There is no difference from that of the comparative group. |
| 0.5: | Symptom somewhere between ‘0’ and ‘1’ is observed. |
| 1: | Congestion, capillary hemorrhage, and slight pore keratosis and |
| | hypertrophy are observed. |
| 1.5: | Symptom somewhere between ‘1’ and ‘2’ is observed. |
| 2: | Medium degree of pore keratosis and hypertrophy are observed. |
| 2.5: | Symptom somewhere between ‘2’ and ‘3’ is observed. |
| 3: | Serious pore keratosis and hypertrophy, and typical comedones are |
| | observed. |

| | Assessment with | (Comparative case- |
|---|---|---|
| | the naked | embodiment)/comparative |
| Testing material | eye | case × 100 |
| Embodiment 1 | 1.05 | 39.7% |
| Comparative case 1 | 1.74 | |
| Embodiment 2 | 1.01 | 42.9% |
| Comparative case 2 | 1.77 | |
| Embodiment 3 | 1.08 | 40.3% |
| Comparative case 3 | 1.81 | |

TABLE 13

| | Comparison | (Comparative case- |
|---|---|---|
| | of comedones | embodiment)/comparative |
| Testing material | area | case × 100 |
| Embodiment 1 | 1.053 | 36.9% |
| Comparative case 1 | 1.668 | |
| Embodiment 2 | 1.017 | 38.7% |
| Comparative case 2 | 1.658 | |
| Embodiment 3 | 1.067 | 37.4% |
| Comparative case 3 | 1.704 | |

As shown in Tables 12 and 13, it has been verified that the cosmetic materials of the embodiments 1-3 have superior effect of alleviating comedones as compared to that of the cosmetic materials of the comparative cases 1-3.

While not wishing to be bound by any theory, the effect of restraining comedones growth and alleviating comedones is believed to be a result of producing the effects of restraining sebum secretion and alleviating inflammation, and the anti-bacterial effect against P. acnes of cryptotanshinone verified in the above-described experiments. It has been verified, through the experiments for assessing the effects of restraining comedones growth and alleviating comedones using ear of rabbit, that the cosmetic material containing cryptotanshinone is remarkably effective in prevention and alleviation of acne. Accordingly, the following experiment has been carried out to verify that the cosmetic material according to the present invention is effective in alleviation of acne in a clinical testing.

Assessment of Effect of Acne Alleviation

To observe the effect of acne alleviation of the cosmetic material according to the present invention, a blind test was carried out with one hundred twenty healthy grown-up men and women at the age of 20-30, having acne.

One hundred twenty men and women were classified into six groups of twenty at random. The cosmetic materials of the embodiments 1-3 and the comparative cases 1-3 were respectively put in same receptacles and the subjects in each group use each cosmetic material twice a day for two months. The assessment was made every two weeks for two months by the survey of subject's opinion and the opinion with the naked eye of two dermatologists regarding the improvement or exacerbation of acne. The results are shown in Table 14.

TABLE 14

| | | Slightly | | |
|---|---|---|---|---|
| | Effective | effective | Unaware | Ineffective |
| Testing material | (persons) | (persons) | (persons) | (persons) |
| Embodiment 1 | 10/20 | 6/20 | 3/20 | 1/20 |
| Comparative case 1 | 0/20 | 2/20 | 2/20 | 16/20 |
| Embodiment 2 | 13/20 | 7/20 | 0/20 | 0/20 |
| Comparative case 2 | 0/20 | 2/20 | 3/20 | 15/20 |
| Embodiment 3 | 12/20 | 6/20 | 2/20 | 0/20 |
| Comparative case 3 | 0/20 | 2/20 | 1/20 | 17/20 |

Referring to Table 14, in case of the cosmetic materials of the embodiments 1-3, ‘effective’ and ‘slightly effective’, which can be attributed to the materials having the effect of alleviating acne, make up 80-100% of the opinions, while 10% or so in case of the cosmetic materials of the comparative cases 1-3 not containing cryptotanshinone. Accordingly, the results show that the cosmetic material according to the present invention is very effective in alleviation of acne.

As described above, the cosmetic material according to the present invention can be safely used without any side effect on skin and the same is very useful for preventing and treating acne because the same has the superior ability of controlling the amount of sebum secretion through inhibition activity of 5 α-reductase and the anti-bacterial and anti-inflammation ability.

While the invention has been described in detail and with reference to specific embodiments thereof, it is intended that the specification and examples be considered as exemplary only and it will be apparent to one skilled in the art that the present invention covers modifications and variations of this invention that come within the scope of the appended claims and their equivalents. Therefore, a true scope and spirit of the invention are indicated by the appended claims.

What is claimed is:

1. A cosmetic composition for preventing or treating acne containing cryptotanshinone as an effective component.

2. The cosmetic composition according to claim 1, comprising an amount of cryptotanshinone that is 0.001-10 weight % on the basis of total composition weight.

3. The cosmetic composition according to claim 2, comprising an amount of cryptotanshinone that is 0.001-2 weight % on the basis of total composition weight.

4. The cosmetic composition according to claim 1, further containing material for restraining over-keratinization.

5. The cosmetic composition according to claim 1, further comprising one or more member selected from the group consisting of salicylic acid, retinoic acid, retinoic acid derivative, benzoyl peroxide as the material for restraining over-keratinization.

6. The cosmetic composition according to claim 1, in the form of a toner, lotion, cream, foundation, essence, gel, pack, foam cleanser or soap.
